(19) United States
(12) Patent Application Publication
(10) Pub. No.: US 2014/0336942 A1
(43) Pub. Date: Nov. 13, 2014

(54) Analyzing High Dimensional Single Cell Data Using the T-Distributed Stochastic Neighbor Embedding Algorithm

(71) Applicant: THE TRUSTEES OF COLUMBIA UNIVERSITY IN THE CITY OF NEW YORK, New York, NY (US)

(72) Inventors: Dana Pe'er, New York, NY (US); El-Ad David Amir, New York, NY (US)

(73) Assignee: THE TRUSTEES OF COLUMBIA UNIVERSITY IN THE CITY OF NEW YORK, New York, NY (US)

(21) Appl. No.: 14/101,909

(22) Filed: Dec. 10, 2013

Related U.S. Application Data
(60) Provisional application No. 61/797,608, filed on Dec. 10, 2012.

(51) Int. Cl. G01N 33/483

(52) U.S. Cl. CPC G01N 33/483
USPC 702/19

(57) ABSTRACT

A method for mapping, graphing, and analyzing high-dimensional single cell data based on multiple parameters associated with the cell, including defining a point associated with the cell in a n-dimensional space; combining the point with other points associated with other cells to form a data set; representing the points in the data set in the n-dimensional space; projecting the points in the n-dimensional space onto a lower-dimensional map; and analyzing the features of interest in heterogeneous tissues using the lower-dimensional map.

### CROSS REFERENCE TO RELATED APPLICATION

This application claims priority to U.S. Provisional Patent Application No. 61/797,608, filed Dec. 10, 2012, which is incorporated by reference herein in its entirety.

### STATEMENT REGARDING FEDERALLY SPONSORED RESEARCH

This invention was made with government support under grant number MCB-1149728 awarded by the National Institutes of Health Roadmap Initiative, the NIH Director's New Innovator Award Program through grant number 1-DP2-OD002414-01, and the National Centers for Biomedical Computing Grant 1U54CA 121852-01A1. The government has certain rights in the invention.

### BACKGROUND

Single-cell technologies have been used in uncovering an expansive degree of heterogeneity between and within tissues. Analysis of single-cell data has shed light on different cellular processes, and provided for the study of a large number of parameters in single cells. Mass cytometry can measure many, e.g., 45 parameters simultaneously in individual cells.

However, despite these advances, it can be problematic to visualize such a high number of dimensions in a meaningful manner. Single-cell data is often examined two dimensions at a time in the form of a scatter plot. However, as the number of parameters increases the number of pairs can become overwhelming: a typical mass cytometry dataset will have several hundred combinations. Additionally, a pairwise viewpoint can miss biologically meaningful multivariate relationships that cannot be discerned in two dimensions.

Certain computational tools can address these problems. However, such tools can cluster cells and collapse the populations, which results in the loss of single-cell resolution of the data. Principal component analysis (PCA), another computational tool, has been applied to mass cytometry and can be used to project data into two dimensions. The caveat is that PCA can be a linear transformation that does not faithfully capture nonlinear relationships.

### SUMMARY

The disclosed subject matter provides techniques for mapping, graphing, and analyzing high-dimensional single cell data based on multiple parameters associated with the cell. For example, a method includes defining a point associated with the cell in a n-dimensional space, combining the point with other points associated with other cells to form a data set, representing the points in the data set in the n-dimensional space, projecting the points in the n-dimensional space onto a lower-dimensional map; and analyzing the features of interest in heterogeneous tissues using the lower-dimensional map. The projection can use a nonlinear dimensionality reduction algorithm to map the points in the n-dimensional space onto the lower-dimensional map.

In an exemplary embodiment n parameters related to a single cell can be obtained by performing measurements on a cell. Such parameters can include molecular species, such as gene or protein epitope expression, as well as morphological features extracted from techniques such as microscopy. Each parameter can be directly measured, e.g., using mass cytometry or flow cytometry. Alternatively, the measurements can be obtained from a third party.

Systems and methods according to the disclosed subject matter can allow for applications including, for example and without limitation, mapping healthy and cancerous bone marrow samples, as well as other types of cancerous tissue, including solid tumors. Healthy bone marrow automatically maps into a consistent shape, whereas leukemia samples map into malformed shapes that are distinct from healthy bone marrow and from each other. The disclosed subject matter can also be used to compare leukemia diagnosis and relapse samples, and to identify a rare leukemia population reminiscent of minimal residual disease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated and constitute part of this disclosure, illustrate some embodiments of the disclosed subject matter.

FIG. 1 is a flow diagram describing one embodiment of the method for analyzing single cell data using a non-linear projection to a lower dimensional map in accordance with the disclosed subject matter.

FIG. 2 is a flow diagram describing one embodiment of a method for representing each of a plurality of cells as a point in a high-dimensional space in accordance with the disclosed subject matter.

FIG. 3 is a flow diagram describing a method for projecting each of the plurality of points to a low-dimensional map in accordance with the disclosed subject matter.

FIG. 4 is a block diagram of a system for the analysis of high-dimensional single-cell data in accordance with the disclosed subject matter.

FIG. 5A shows how one embodiment of the disclosed subject matter operates on a synthetic example; an algorithm can identify the global structure of the embedded manifold (a 1D line, along with its curvature, embedded in 3D space) and the local structure (pairwise distances between points along the line are conserved). FIG. 5B shows a map of single cell data for healthy bone marrow cells generated by an exemplary method in accordance with disclosed subject matter. Each point in the map represents an individual cell, and the color of each point represents the immune subtype of the cell. FIG. 5C shows biaxial plots representing the same data shown in FIG. 5B, with select subpopulations shown with canonical markers. FIG. 5D shows the map of FIG. 5B with each cell colored based on CD11b expression.

FIG. 6A shows the map of FIG. 5B with each of the cell subtypes surrounded by a gate corresponding to that subtype's color. FIG. 6B shows maker expression level densities plotted for the entire population, the manually gated CD11b-monocyte cells, and the CD11b-monocyte cells gated in accordance with an exemplary embodiment of the disclosed subject matter.

FIG. 7 shows two maps for different subsamples of the same sample generated using an exemplary method in accordance with the disclosed subject matter.

FIG. 8 shows leave-one-out maps for the same sample generated using an exemplary method in accordance with the disclosed subject matter.

FIG. 9A shows a map of single cell data for healthy bone marrow cells generating using an exemplary method in accordance with the disclosed subject matter omitting various markers from the parameter set. FIG. 9B shows how three non-canonical markers can separate monocytes (red) and B cells (green).

FIG. 10 shows a map of single cell data from separate samples generated using an exemplary method in accordance with the disclosed subject matter. FIG. 10A shows a map with each cell color coded by sample. FIG. 10B shows a map color coded by subtype. FIG. 10C shows a map of single cell data from two healthy donors and two ALL patients.

FIG. 11 shows three maps using different marker panels generating using an exemplary method in accordance with the disclosed subject matter.

FIG. 12 shows a map generated using an exemplary method in accordance with the disclosed subject matter from data obtained using fluorescence-based flow cytometry.

FIG. 13 shows maps of single cell data from samples from cancer patients generated using an exemplary method in accordance with one embodiment of the disclosed subject matter. FIG. 13A shows contour plots of the maps from each of four cancer samples: two from ALL patients and two from AML patients. FIG. 13B shows a map of an AML patient.

FIG. 14 shows additional maps for the AML patient shown in FIG. 13B generated using an exemplary method in accordance with the disclosed subject matter.

FIG. 15 shows maps of single cell data for diagnosis and relapse samples generating using an exemplary method in accordance with one embodiment of the disclosed subject matter. FIG. 15A shows a contour plot of the map combining diagnosis and relapse samples. FIG. 15B shows the same map with cells from both samples colored by marker expression levels.

FIG. 16 shows additional maps of diagnosis and relapse samples from the AML patient with cells colored by the expression level indicated in the panel title.

FIG. 17 shows maps of diagnosis and relapse samples of an additional AML patient generated using an exemplary method in accordance with the disclosed subject matter.

FIG. 18 shows a map of single cell data generated using a MRD detection procedure in accordance with one embodiment of the disclosed subject matter. FIG. 18A shows a map generated using a MRD sample including a healthy sample spiked with ALL cells. FIG. 18B shows the density of the cells in the suspect region (shown in red) and the non-suspect region (shown in cyan). FIG. 18C shows the map of FIG. 18A, with the cells color coded by healthy barcode (cyan) or ALL barcode (red).

FIG. 19 shows a map of single cell data generated using a MRD detection procedure in accordance with one embodiment of the disclosed subject matter, without the addition of the ALL samples.

FIG. 20 shows a map of single cell data generated using a MRD detection procedure in accordance with one embodiment of the disclosed subject matter, using a different marker panel than the one used in connection with FIG. 18.

FIG. 21 is an example of the method applied to flow cytometry data collected from a clinical bone marrow sample of MRD in the ALL context.

FIG. 22 shows the method applied to clinical MRD bone marrow samples collected using flow cytometry.

FIG. 23 shows the method applied to mass cytometry data collected from a melanoma derived cell line.

FIG. 24 shows the method applied to map heterogeneity in primary ovarian cancers.

FIG. 25 shows the method applied to mass cytometry data collected from a glioblastoma sample. FIG. 25A shows the single cell expression of four reported TIC markers on 100,000 cells in accordance with an embodiment the disclosed subject matter. FIG. 25B shows basal levels of p-Rb and p-S6 correlated with Sox2 and CD44 marker expression. FIG. 25C sows a heat plot with each square representing a fold-change of the indicated marker in the cells in that region of the plot.

### DETAILED DESCRIPTION

The disclosed subject matter is generally directed to a method for analyzing high-dimensional data by projecting the high-dimensional data onto a low-dimensional map. A plurality of points can be mapped from a high dimensional space to the low-dimensional space using a nonlinear dimensionality reduction algorithm such as the t-Distributed Stochastic Neighbor Embedding (t-SNE) algorithm. The resulting map can be used to analyze developmental systems in healthy and diseased cells and identify rare subpopulations of cells within a sample.

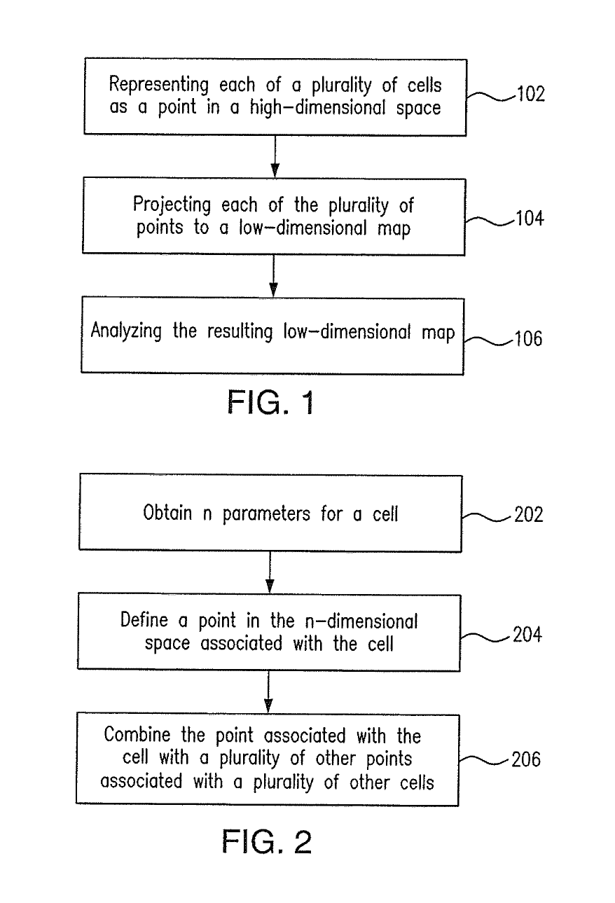

One embodiment of the method for analyzing high-dimensional single cell data in accordance with the disclosed subject matter is shown in FIG. 1. Each of a plurality of cells can be represented as a point in a high-dimensional space at 102. As used herein, the term “high-dimensional space” refers to a conceptual space having a large number of dimensions. In accordance with one embodiment of the disclosed subject matter, the term “high-dimensional space” can refer to a conceptual space having four or more dimensions, five or more dimensions, six or more dimensions, seven or more dimensions, eight or more dimensions, nine or more dimensions, ten or more dimensions, fifteen or more dimensions, or twenty or more dimensions. For example, a conceptual space having 45 dimensions is a “high-dimensional space.” Reference will be made herein to a n-dimensional space, which is a high-dimensional space where n is four or higher.

An exemplary embodiment of a method for representing each of a plurality of cells as a point in a n-dimensional space in accordance with the disclosed subject matter is illustrated in FIG. 2. First, n parameters related to a single cell can be obtained at 202. The parameters can be obtained by performing measurements on a cell. For example, each parameter can be directly measured. Any measuring technique can be used as known in the art. For example, each parameter can be measured using mass cytometry or flow cytometry. Alternatively, the measurements can be obtained from a third party.

These measurements can relate to any type of cell. In accordance with one embodiment, the cell is a bone marrow cell. In another embodiment, the cell can be a cancerous cell such as a cell from an Acute Lymphoblastic Leukemia (ALL) patient or an Acute Myeloid Leukemia (AML) patient, as well as a cell from solid tumors such as ovarian cancer or glioblastoma.

The parameters measured in accordance with the disclosed subject matter can be surface markers. For example, one parameter can be the expression level of one protein. In accordance with another embodiment of the disclosed subject matter, the parameters can be functional markers, such as those that probe signaling, cell cycle, and metabolism.

In accordance with one embodiment of the disclosed subject matter, exactly n parameters are measured. That is, the n-dimensional space corresponds to the complete panel of markers associated with each cell. In accordance with another embodiment, m parameters are measured, where m>n. The n parameters utilized in connection with this method can then be chosen from the m measured parameters. The n parameters can be chosen to suit a particular purpose. For example, where only n of the m parameters are related to a feature of interests, those n parameters can be used in connection with the disclosed methods.

With further reference to FIG. 2, a point associated with the cell can be defined in the n-dimensional space at 204. The point can be defined based on the n parameters obtained at 202. Each point in the n-dimensional space will have n coordinates. Thus, the point x associated with the cell can be defined as:

x=(p₁,p₂, . . . pₙ₋₁,pₙ) (1)

where pi is one of the obtained parameters for each i between 1 and n. Thus, p₁ can be the expression level of protein 1, p₂ can be the expression level of protein 2, and so on.

With further reference to FIG. 2, the point associated with the cell can be combined with a plurality of other points associated with a plurality of other cells to form a data set at 206. The data set represents a plurality of points plotted in a n-dimensional space.

With further reference to FIG. 1, each of the plurality of points in the n-dimensional space can be projected to a low-dimensional map at 104. As used herein, the term “low-dimensional” refers to two-dimensional or three-dimensional.

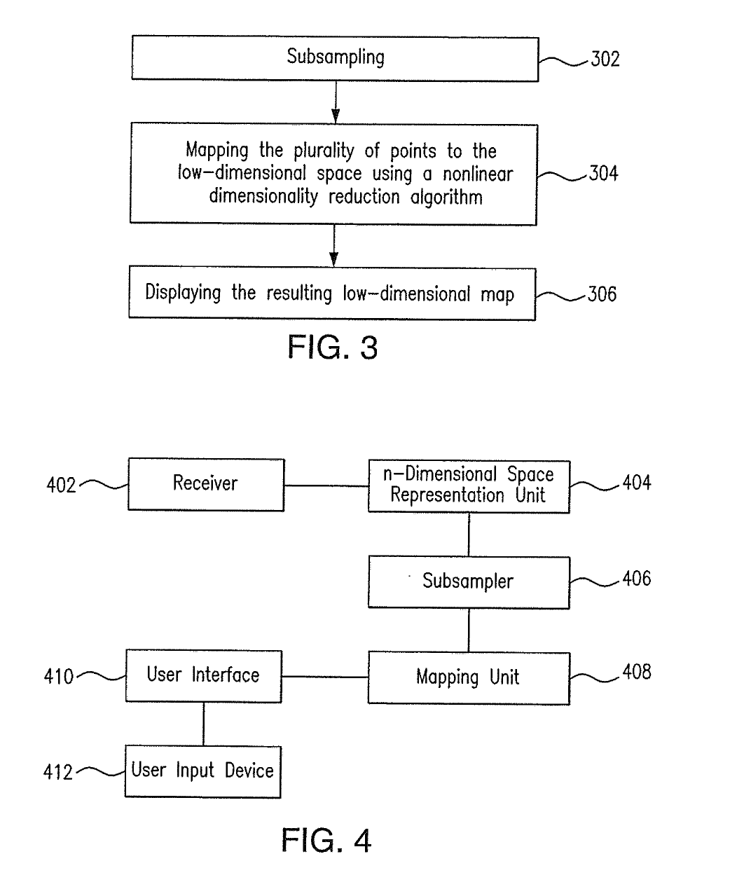

One embodiment of the method for projecting each of the plurality of points in the n-dimensional space to a low dimensional map is illustrated in FIG. 3. First, the plurality of points in the data set can be subsampled at 302. In particular, where there are a large number of points, a crowding problem can occur where distant points in the high-dimensional space collapse onto nearby areas of the low dimensional map and form one large, dense region with no separation between populations. Therefore, the data set can be subsampled and only the sampled data points can be used. The subsampling can be done randomly.

With further reference to FIG. 3, the plurality of points can be mapped from the high dimensional space to the low-dimensional space using a nonlinear dimensionality reduction algorithm 304. For example, and in accordance with one embodiment of the disclosed subject matter, the nonlinear dimensionality reduction algorithm can be the t-Distributed Stochastic Neighbor Embedding (t-SNE) algorithm. For ease of reference, the use of the t-SNE algorithm in connection with the disclosed subject matter will be referred to as viSNE.

First, a pairwise similarity matrix P is calculated in the n-dimensional space. For each point xi corresponding to one of the plurality of points in the n-dimensional space, the similarity of xᵢ to xⱼ can be defined as:

$${P}_{j / i} = \frac{exp ( - {‖ {x}_{i} - {x}_{j} ‖}^{2} / 2 {σ}_{i}^{2} )}{{\sum }_{k i} exp ( - {‖ {x}_{i} - {x}_{j} ‖}^{2} / 2 {σ}_{i}^{2} )} \qquad ( 2 )$$

where σᵢ is the variance of xᵢ.

For each xᵢ, a binary search can be performed to identify the value of σᵢ that produces a Pᵢ with a fixed Perplexity. The fixed Perplexity can be defined to suit a particular purpose. In one embodiment, the fixed Perplexity is provided by a user. The Perplexity can be defined as:

Perp(Pᵢ)=2^{H(P}^{ⁱ}⁾ (3)

where H(Pᵢ) is the Shannon's entropy and can be defined as:

$$H ( {P}_{i} ) = - {\sum }_{j} {P}_{j \vert i} {log}_{2} {P}_{j \vert i} \qquad ( 4 )$$

The joint similarity of xᵢ and xⱼ can be defined as:

$${P}_{ij} = \frac{{P}_{j \vert i} + {P}_{i \vert j}}{2 n} \qquad ( 5 )$$

A starting position for each of the plurality of points in the low-dimensional space, Y_{—}0 can then be randomized. The position of each of the plurality of points in the low dimensional-space can then be iteratively updated. In iteration i, the similarity matrix in the low-dimensional space, Q, can be calculated according to the points' current position, Y_{i−1}.

For each pair of points in the low-dimensional space, yi and yj, the similarity can be defined as:

$${Q}_{ij} = \frac{{( 1 + {‖ {y}_{i} - {y}_{j} ‖}^{2} )}^{- 1}}{{{Σ}_{k \neq 1} ( 1 + {‖ {y}_{i} - {y}_{j} ‖}^{2} )}^{- 1}} \qquad ( 6 )$$

The t-SNE algorithm can minimize the Kullback-Leibler (KL) divergence between the joint probability distribution P in the high dimensional space and the joint probability distribution Q in the low dimensional space. The KL divergence can be defined as:

$$KL ( P \vert \vert Q ) = {\sum }_{i} {\sum }_{j} {P}_{ij} log \frac{{P}_{ij}}{{Q}_{ij}} \qquad ( 7 )$$

Gradient descent can then be used to calculate the new position of each point, Y_{—}1, in order to minimize the divergence between the pairwise similarity matrix in the n-dimensional space, P, and the similarity matrix in the low-dimensional space, Q. The gradient of the KL divergence between P and Q can be defined as:

$$\frac{δC}{δ {y}_{i}} = 4 {\sum }_{j} ( {P}_{ij} - {Q}_{ij} ) ( {y}_{i} - {y}_{j} ) {( 1 + {‖ {y}_{i} - {y}_{j} ‖}^{2} )}^{- 1} \qquad ( 8 )$$

The t-SNE algorithm is more fully described in “Visualizing Data using t-SNE” by Laurens van der Maaten and Geoffrey Hinton, Journal of Machine Learning Research 9 (2008) 2579-2605, which is incorporated herein by reference for all purposes.

With further reference to FIG. 3, the resulting low-dimensional map can be displayed at 306. The low-dimensional map can be displayed using any method for displaying two or three dimensional data as known in the art. For example, a two-dimensional map can be displayed as a scatter plot. A three-dimensional map can be displayed on a computer monitor. Additional information can be added to the map through the use of color, which allows for supplemental information to be added for purposes of analysis. Color can be used to interactively visualize features of the sampled cells as described below with reference to the examples.

With further reference to FIG. 1, the resulting low-dimensional map can be analyzed at 106. The low-dimensional map can be used for a variety of purposes as described below with reference to the Examples. In general, the disclosed subject matter can be used to map developmental systems in healthy and diseased patients. For example, the disclosed subject matter can be used to map healthy developments for stem cells.

The resulting low-dimensional map can be used to identify rare subpopulations of cells. In one embodiment, the disclosed subject matter can be used to identify drug resistant subsets. For example, a drug can be applied to a collection of cells and a map can be generated. The map can be used to identify drug resistant cells. The cells can be sorted and studied.

In accordance with another embodiment, the disclosed subject matter can be used to diagnose cancer. For example, the disclosed subject matter can be used as an early detection method due to its ability to identify rare subpopulations of cells. In accordance with another embodiment, the disclosed subject matter can be used to detect recurrence of cancer in patients that were previously diagnosed with cancer (i.e., to detect Minimal Residual Disease).

The disclosed subject matter further includes a system for analyzing high-dimensional single cell data. For purpose of explanation and illustration, and not limitation, an exemplary embodiment of the system for analyzing high-dimensional single cell data in accordance with the disclosed subject matter is shown in FIG. 4. The system includes a receiver 402, a high-dimensional space representation unit 404, a subsampler 406, a mapping unit 408, and a user interface 410.

The receiver is configured to obtain n parameters related to a single cell. In accordance with one embodiment of the disclosed subject matter, the receiver can be coupled to a measurement device that captures the relevant parameters directly. In another embodiment, the receiver can be coupled to a communications network such as the Internet to receive the parameters from a third party source. In accordance with another embodiment, the receiver can be coupled to a user input device 412, such as a keyboard.

The receiver 402 can be coupled to a high-dimensional space representation unit 404. As used herein, the term “coupled” means operatively in communication with each other, either directly or indirectly, using any suitable techniques, including hard wire, connectors, or remote communication. For example, the receiver 402 can be coupled to the high-dimensional space representation unit 404 through a hard drive or other data storage unit. The high-dimensional space representation unit 404 is configured to define a point in the n-dimensional space associated with the cell and combine the point associated with the cell with a plurality of other points associated with a plurality of other cells, as further described herein.

The high-dimensional space representation unit 404 can be coupled to a subsampler 406. The subsampler 406 can be configured to subsample the data set as necessary and appropriate, as discussed herein.

The subsampler 406 can be coupled to a mapping unit 408. The mapping unit 408 is configured to map the plurality of points in the high-dimensional space to the low-dimensional space using a nonlinear dimensionality reduction algorithm as described herein. The nonlinear dimensionality reduction algorithm can be the t-Distributed Stochastic Neighbor Embedding (t-SNE) algorithm.

The mapping unit 408 can be coupled to a user interface 410. The user interface 410 is configured to display the resulting low-dimensional map. The user interface 410 can be, for example, a computer monitor. The user interface 410 can further be coupled to a user input device 412 to allow the user to manipulate the data set. For example, the user input device 412, such as a keyboard and/or a computer mouse, can allow the user to specify which parameters should be used in connection with the disclosed subject matter. Additional functional units can be used to perform other functions of the method as disclosed herein.

For example, each of the functional units can be implemented using an integrated single processor. Alternatively, each functional unit can be implemented on a separate processor. Therefore, the single-cell data analysis system can be implemented using at least one processor and/or one or more processors.

The at least one processor can include one or more circuits, which can be designed so as to implement the disclosed subject matter using hardware only. Alternatively, the processor can be designed to carry out the instructions specified by computer code stored in a hard drive, a removable storage medium, or any other storage media. Such non-transitory computer readable media can store instructions that, upon execution, cause the at least one processor to perform the methods as disclosed herein.

### Examples

### Human Haematopoiesis in Bone Marrow

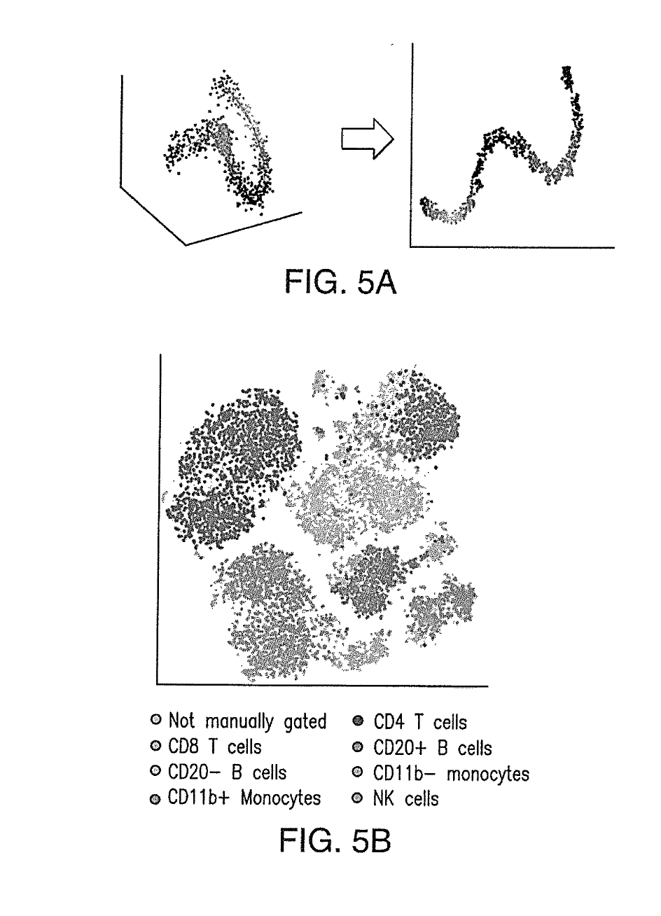

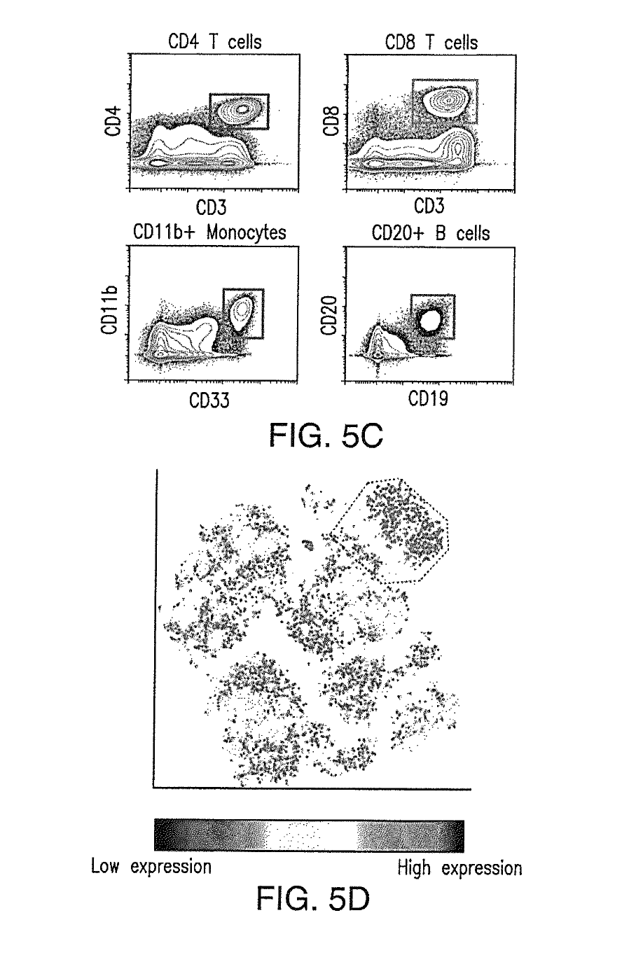

Mass cytometry was used to measure a panel of surface markers in healthy bone marrow. viSNE was applied to Marrow1, a sample from this data. The resulting map is shown in FIG. 5B. Each point in the two-dimensional map represents an individual cell. To validate the map, an independently-derived labeling of the cells with classic immune subtypes, based on manual gating: a succession of gates drawn on biaxial plots two markers at a time, was utilized. The color of each individual point represents the immune subtype of the cell based on independent manual gating. While viSNE was not provided with these labels or any knowledge of immune subsets, it grouped cells in the same subsets together and separated the subsets from one another. viSNE accurately distinguished CD4 and CD8 T cells, mature and immature B cells, mature and immature monocytes and NK cells. Notably, NK cells formed a distinct subset even though CD56, their canonical marker, was not included in the panel.

FIG. 5D shows the same map that is shown in Figure SB with each point colored based on CD11b expression. Many of the cells were not labeled as monocytes by manual gating. When cells were colored based on their marker expression levels, it was revealed that the monocytes self-organized based on a gradient of CD11b (a marker indicating monocyte maturity). This highlights the continuous and gradual nature of CD11b expression during monocyte maturation.

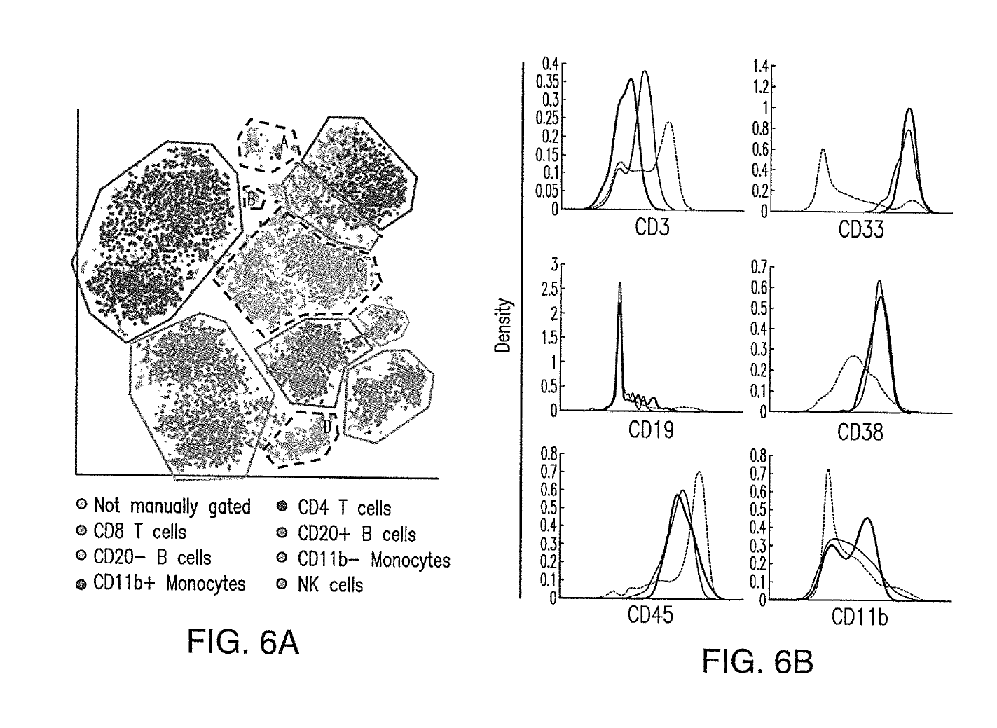

Traditional gating relies on hard thresholds to classify cells into subtypes. However, cells whose marker values are slightly below the threshold might not be labeled correctly, or labeled at all. To compare between the expert gating and the map, the regions corresponding to the different subtypes in the map were gated. The resulting map is shown in FIG. 6. In all cases, the present gate included cells that were not labeled, but that belonged to the respective cell type based on their marker expression. For example, in FIG. 5D, the gated region contains CD33+ cells (canonical monocyte marker). Only 47% of these cells were labeled as monocytes by the manual gating (FIG. 5B). The marker intensity distributions between the CD11b− monocytes in the present gate were compared to the manual gate (FIG. 6B). The distributions are similar, supporting the notion that these are indeed CD11b-monocytes. The disclosed subject matter can take into account all phenotypic markers concurrently instead of relying on hard thresholds and as a result, can both label more cells and capture a more accurate view of the cell variability within each subtype.

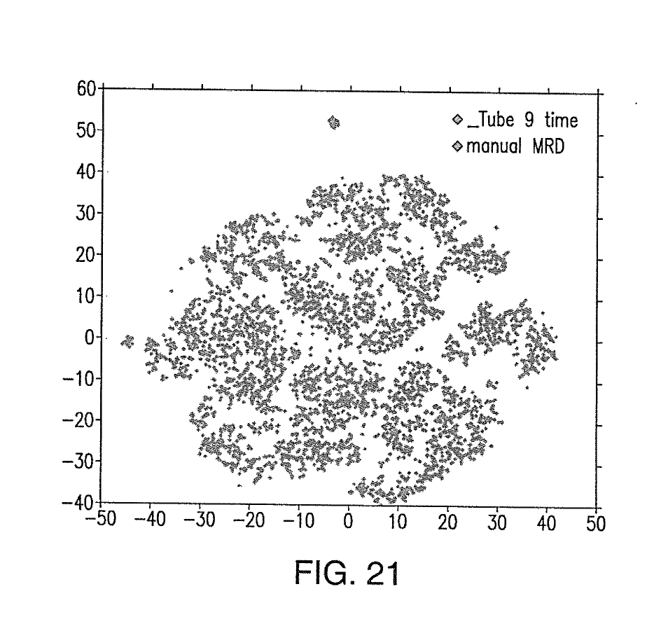

FIG. 21 shows an application of the disclosed method to flow cytometry data collected from a clinical bone marrow sample of Minimal Residual Disease (MRD) in the ALL context. Healthy cells are colored magenta and the recurrent abnormal ALL cells in the sample are cyan, demonstrating that the disclosed method effectively separates healthy and MRD cells in a clinical setting.

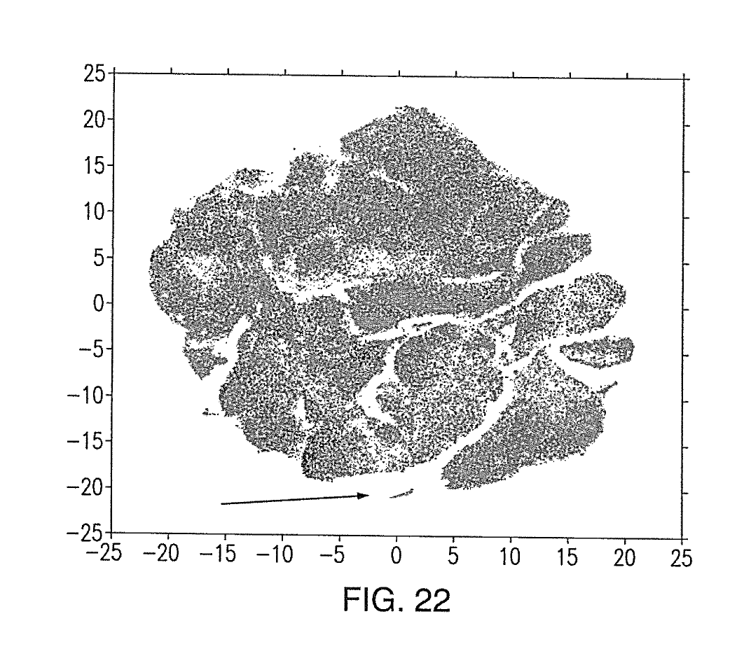

FIG. 22 shows is another demonstration of clinical MRD samples, collected using flow cytometry. The plot includes data from ten healthy bone marrows, superimposed with one suspected MRD patient. All healthy cells, from all samples, mix well among themselves, except for the abnormal MED cells in red (pointed to by the arrow), which are separated from the normal cells. This separation allows clinicians to identify the abnormal cells without the need for pathologist labeling.

### Consistency and Robustness

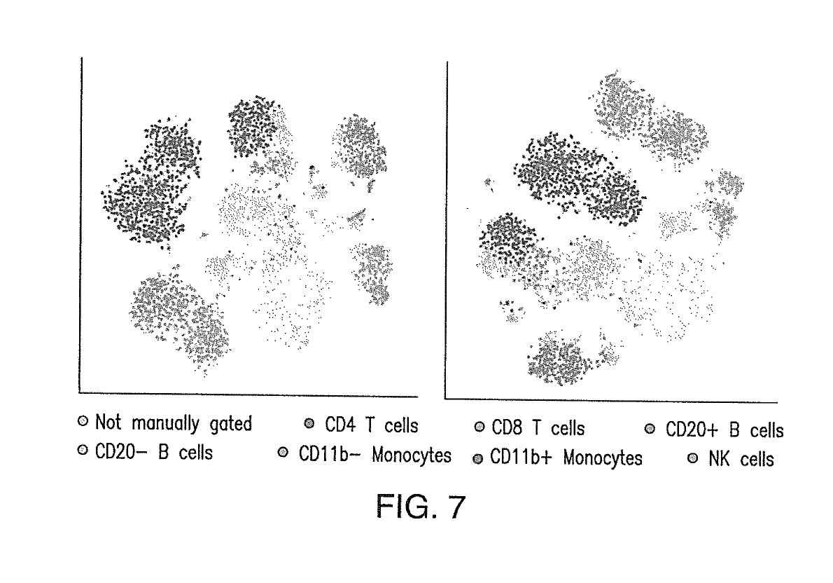

A number of analyses were performed to evaluate the robustness of viSNE. FIG. 7 shows two maps related to the Marrow1 sample. However, each map is based on a different subsample of 6,000 cells from Marrow1. The multiple runs of viSNE on the same data provide similar maps: the separation between identifiable subsets is conserved. In particular, the healthy subtypes are similarly separated, with the same subpopulations identified.

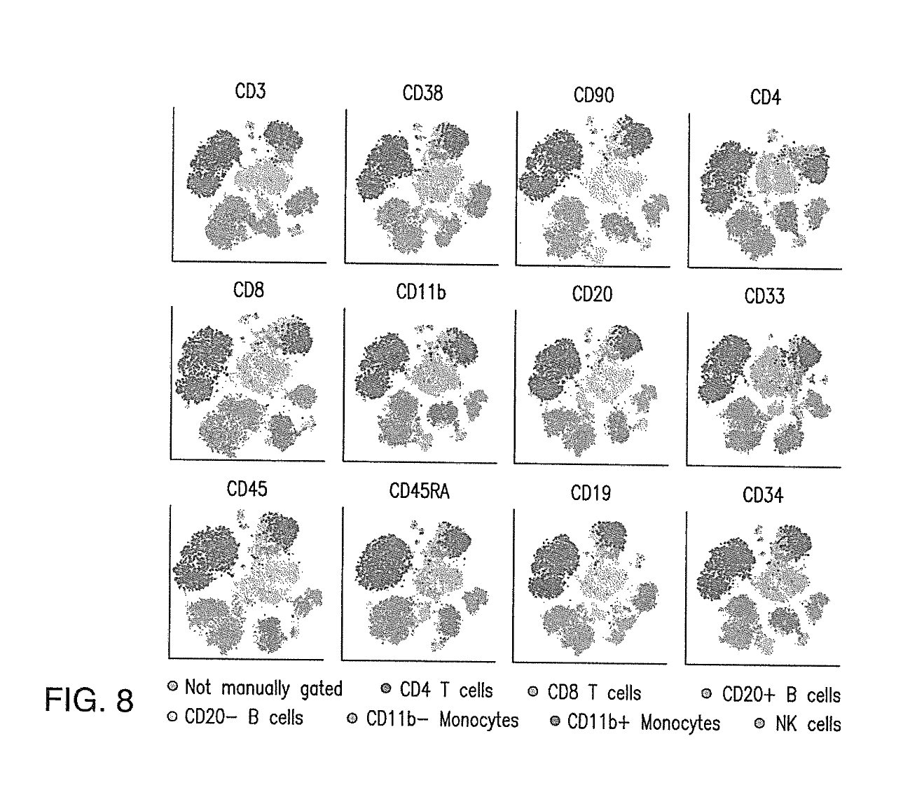

To test viSNE's reliance on the specific markers in the panel, viSNE was run multiple times, excluding some of the markers in each run. FIG. 8 shows leave-one-out maps of Marrow1. The cells are the same cells that are shown in FIG. 5B. Each panel is a map with twelve markers. The marker in the panel's title has not been used for that run. The resulting map remains consistent following the removal of each single marker. Despite removing markers, subtypes are identified and separated correctly in each panel. The different maps are almost identical to each other.

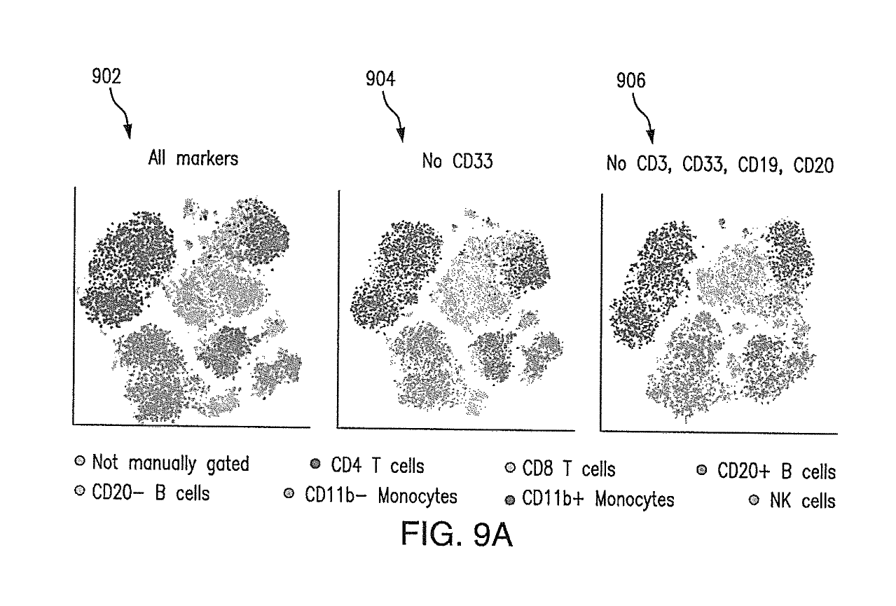

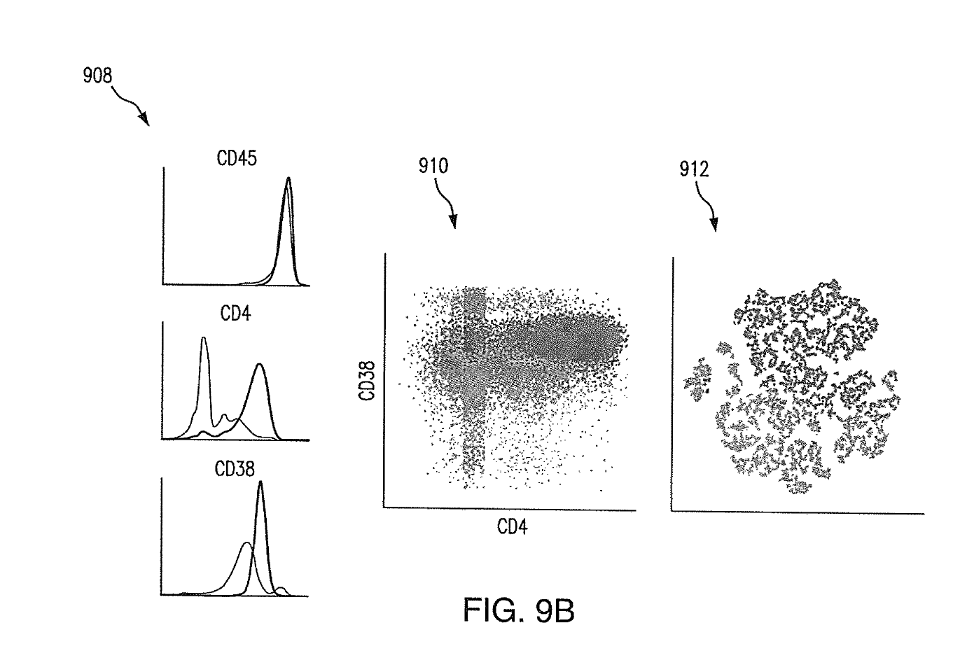

FIG. 9A provides further evidence that viSNE does not rely on a single marker. In particular, map 902 is the same map shown in FIG. 5B, and includes all 13 markers. Map 904 shows a map of the same cells after removing CD33. Map 904 is very similar to map 902 and still identified monocytes. Map 906 shows a map of the same cells after removing CD33, CD3, CD 19, and CD20. Even when removing the four canonical markers for B cells, T cells and myeloid cells (CD19/CD20, CD3 and CD33, respectively), the map remains consistent with the one constructed with all thirteen markers.

A toy example was created from the Marrow1 data to better understand how non-canonical markers can encode information typically derived from canonical markers. Myeloid cells and B cells (their canonical markers are CD33 and CD19/CD20, respectively) were manually gated. Three non-canonical markers were chosen to distinguish between the two cell types: CD38, CD4 and CD45. The distributions of each marker are shown in 908. The x-axis represents marker intensity and the y-axis represents density of cells at this intensity. When examining the marker distributions of these populations, one marker at a time or in pairs of markers, the populations overlap. A biaxial scatter plot 910 demonstrates the overlap of the populations of CD4 (x-axis) and CD38 (y-axis) in two dimensions.

Although the populations of CD4 and CD38 overlap almost entirely in CD45, the use of a second dimension allows viSNE to separate the monocytes and the B cells due to the fact that each subset resides on different manifolds in 3D. Indeed, viSNE effectively distinguishes between the manifolds and separates the two cell types almost perfectly, as shown in map 912.

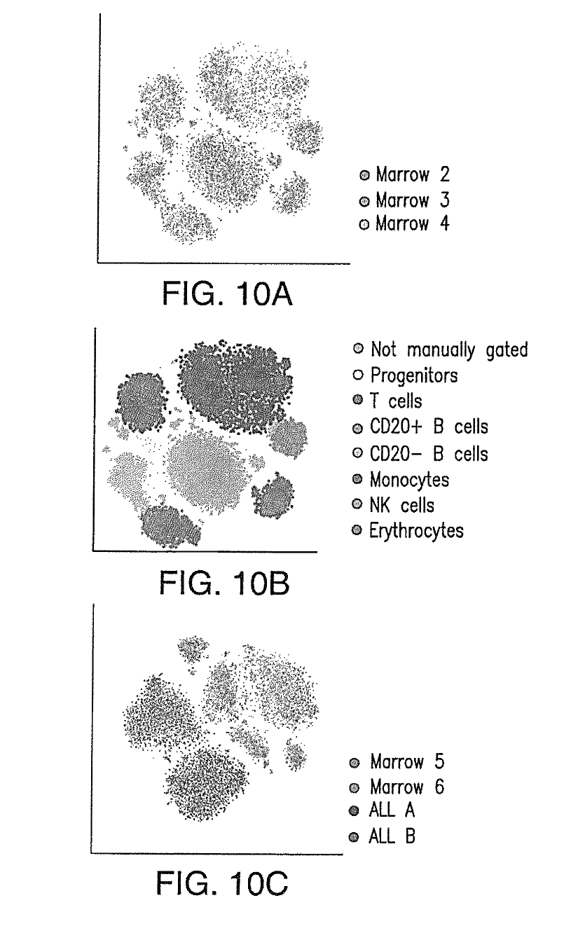

In order to examine viSNE's robustness when applied to multiple healthy individuals, mass cytometry was used to collect data from three healthy bone marrow samples. A panel of 31 phenotypic markers was used. The resulting viSNE map demonstrates both the consistency between healthy samples and viSNE's ability to handle higher dimensionality. FIG. 10A illustrates the resulting map with coloring representing the sample (i.e., the individual from which each cell was obtained). As shown, the cells are grouped into distinct subpopulations and cells from all three individuals overlap within each subpopulation.

FIG. 10B illustrates the same map color coded by subtypes as identified by marker expression level. Mostly the same subpopulations identified in Marrow1 are found, and each corresponding subtype shares a similar shape (e.g. B cells and monocytes).

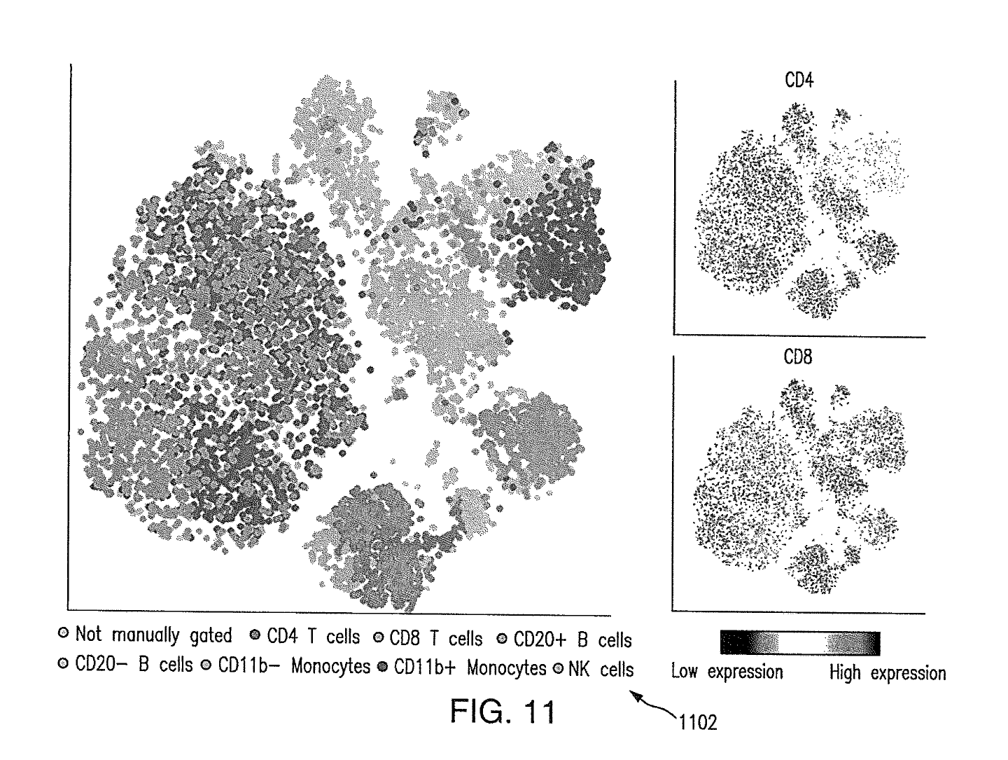

Differences in the marker panel led to minor differences in the populations identified. For example, FIG. 11 illustrates the map of the Marrow 1 sample with CD4 and CD8 omitted. In map 1102, CD4+ and CD8+T cells were merged into one population, as neither of these markers was measured. However, information in the other channels allows for a partial separation between them. New subpopulations identified in this panel include progenitors (CD34/CD117) and erythrocytes (CD61).

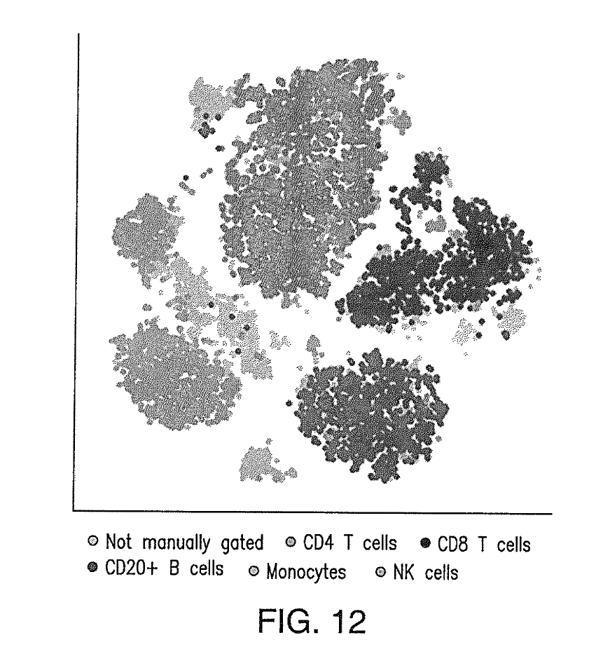

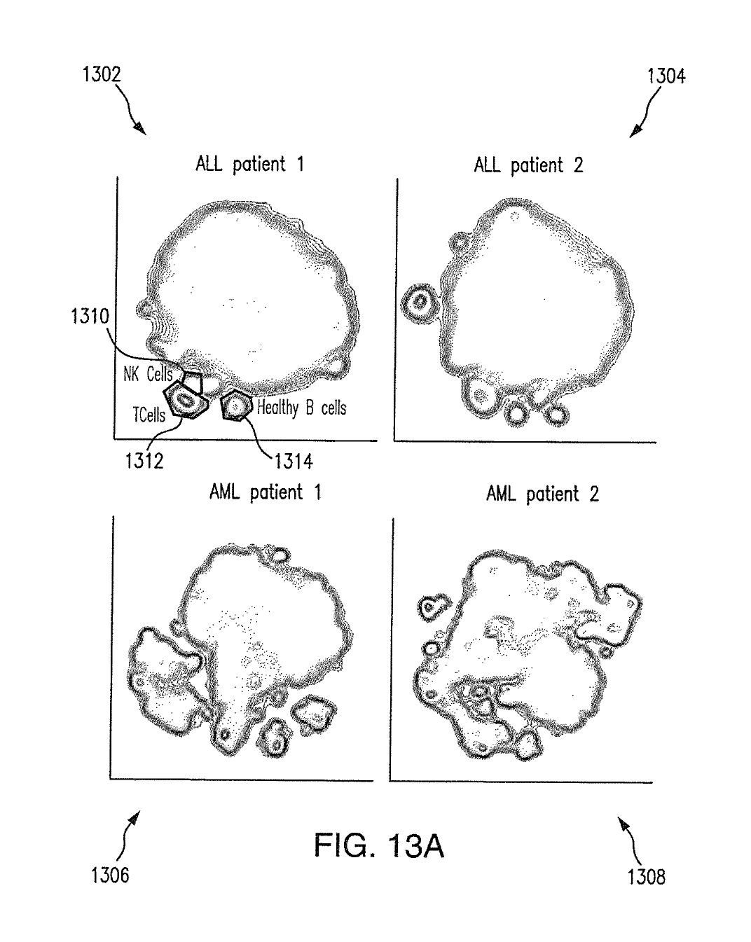

A bone marrow sample was collected using conventional fluorescence-based flow cytometry. The flow cytometry panel included eight markers. viSNE was then applied to the bone marrow sample. The resulting map is shown in FIG. 12. The map in FIG. 12 is similar to the map in FIG. 5B. Due to the lower number of markers, a lower number of subtypes was identified.

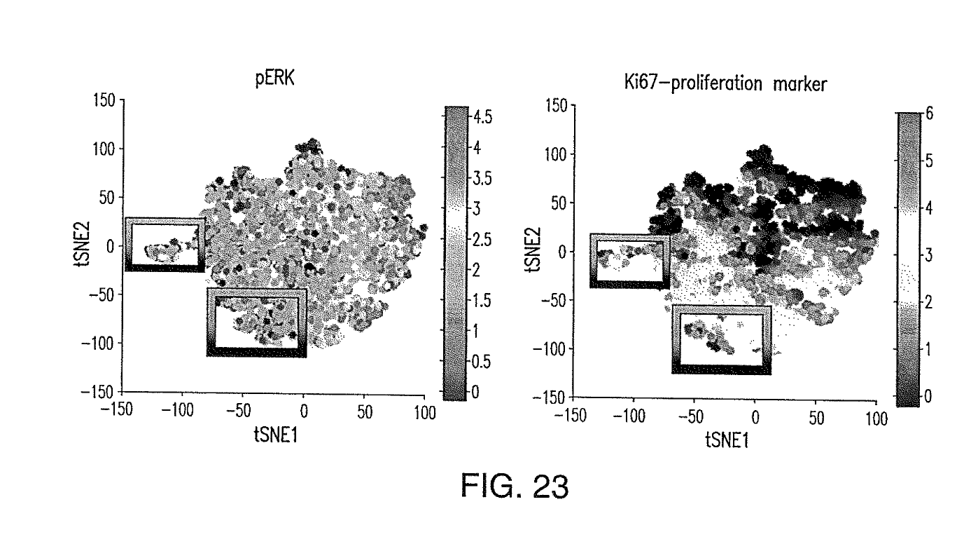

FIG. 23 shows mass cytometry data collected from a melanoma derived cell line following treatment with drug (a BRAF inhibitor in clinical use, Vemurafenib). Mapping is based on levels of internal phosphorelated protein epitopes. The map shows populations with different responses to the drug. Ki67 maps proliferating cells, those cells that continue to proliferate even though a high dose of the drug is applied. Those cells that continue to proliferate are distinguishable from cells that continue to express pERK. pERK is considered a regulator of proliferation in melanoma and is the downstream target of the Vemurafenib drug. The mapping provides insight into the mechanisms of drug resistance in this tumor.

### Cancer Heterogeneity

viSNE to explore the less charted territory of cancer heterogeneity. Four bone marrow samples were obtained, two donated from healthy individuals and two from Acute Lymphoblastic Leukemia (ALL) patients. A 29 marker panel was used. The resulting map is illustrated in FIG. 10C. Each cell is color coded based on sample. Similar to FIG. 10A, the two healthy samples (Marrow5 and Marrow6) overlap and map together. In contrast, the two cancer samples (ALL A and ALL B) occupy a completely separate region within the map, and these are also separated from each other, with each forming a distinct population. A small amount of the cells from the ALL samples (˜5%) overlap with the healthy cells. Inspection of these cells reveals marker combinations that correspond to healthy immune cells, supporting their placement with healthy cells.

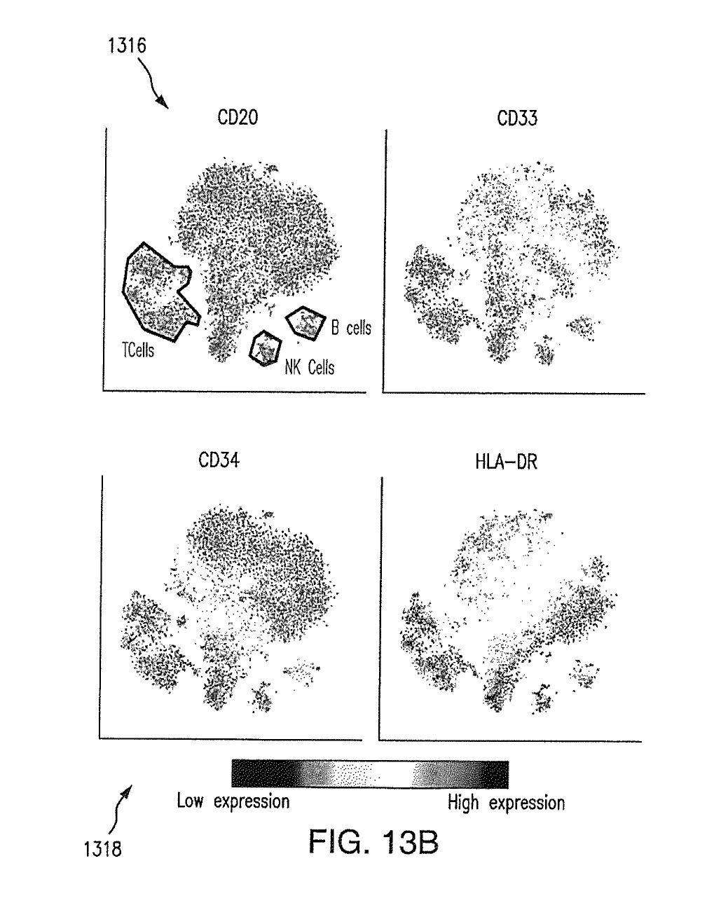

Applying viSNE to each ALL sample separately provides more resolution and detail for each cancer. Contour plots of each of four cancer samples are shown in FIG. 13A. Plots 1302 and 1304 are related to ALL patients, while plots 1306 and 1308 are related to Acute Myeloid Leukemia (AML) patients. The contour lines represent cell density in each region of the map. Each map has a single large population and a number of small separated subpopulations. Each cancer mapped into a single large deformed shape. The small populations are healthy immune subtypes as identified by their marker expression combination. For example, in plot 1302, three healthy subtypes 1310, 1312, and 1314 are highlighted. It is observed that while the maps of healthy samples are comparable, each cancer forms a unique map, demonstrating heterogeneity between and within patient samples.

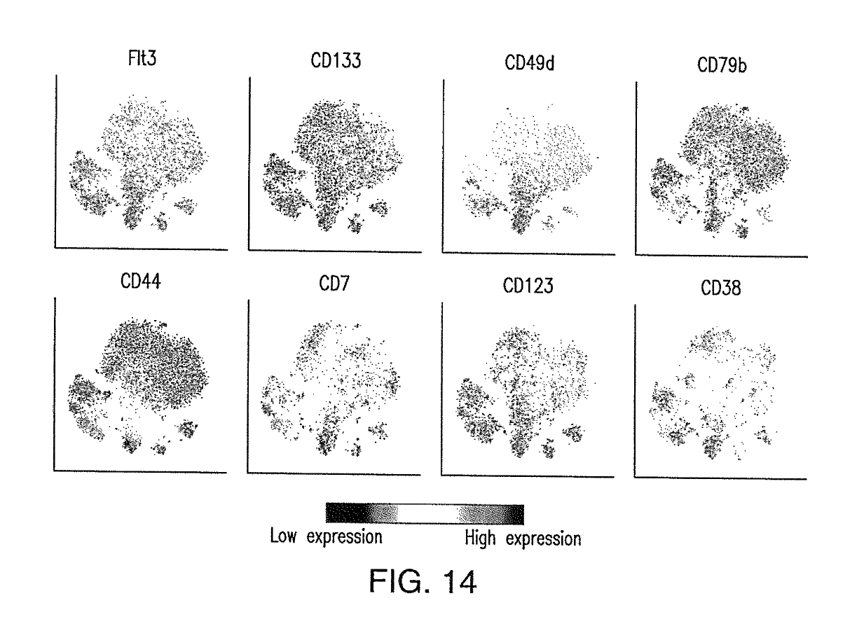

A map for an AML patient is shown in FIG. 13B. Cells are colored by marker expression levels. CD20 helps identify the healthy B cell subpopulation 1316. The other markers—CD33, CD34 (hematopoietic progenitor cells), and HLA-DR (MHC class II, typically expressed on B cells and monocytes)—form clear gradients on the map (blue to red) in different regions and directions. Additional characterization of the map of AML1 is illustrated in FIG. 14. Cells are colored by the expression levels of the marker in the panel's title. Most markers follow one of two behaviors: clustered (such as CD79b) or gradient (such as CD7).

One of the most dominant gradients is CD34, a stem cell and progenitor marker in healthy hematopoiesis. The gradient for CD34 is shown in map 1318. Within the highly expressing CD34 cells (immature) there is a CD33 gradient (indicating differentiation into monocytes) without the attenuation of CD34 that occurs during healthy development. These gradients suggest a derailed developmental program resulting in abnormal phenotypic states (combinations of phenotypic markers) that express progenitor markers (CD34) concurrently with differentiation markers (CD33). While not intending to be bound by any theory of operation, one hypothesis is that a progenitor-like state (associated with CD34) is enforced by oncogene activity, and attempted differentiation (rise of CD33) is stunted. The data exhibits a heterogeneous spectrum of aberrant phenotypic states and a continuum of intermediate states between them.

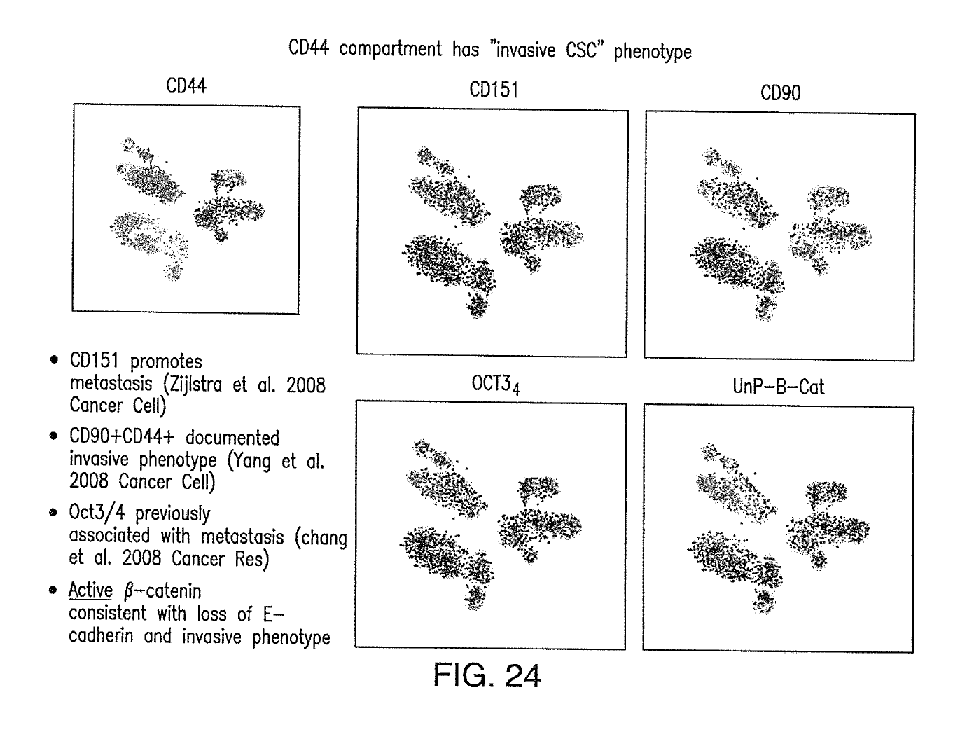

FIG. 24 maps heterogeneity in primary ovarian cancers patients. This heterogeneity identifies different subpopulations of the tumor that differ in their “stem-ness” (ability to reseed a tumor) and ability to metastasize. This is application of the disclosed method on solid tumors using internal markers.

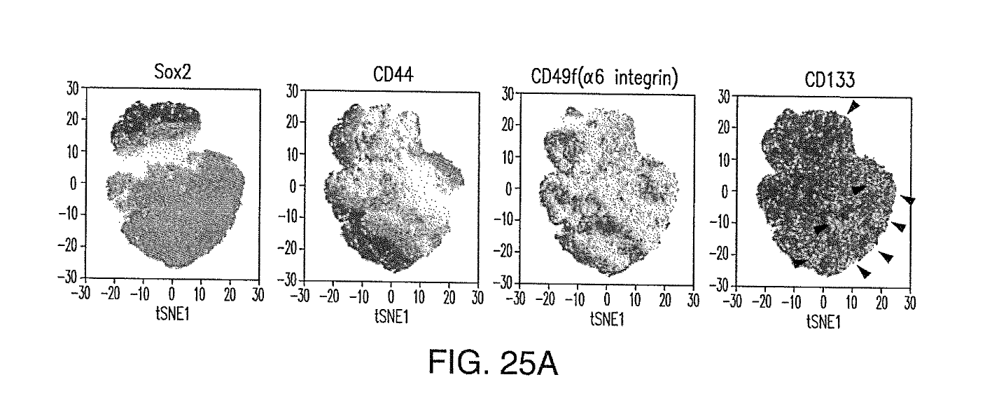

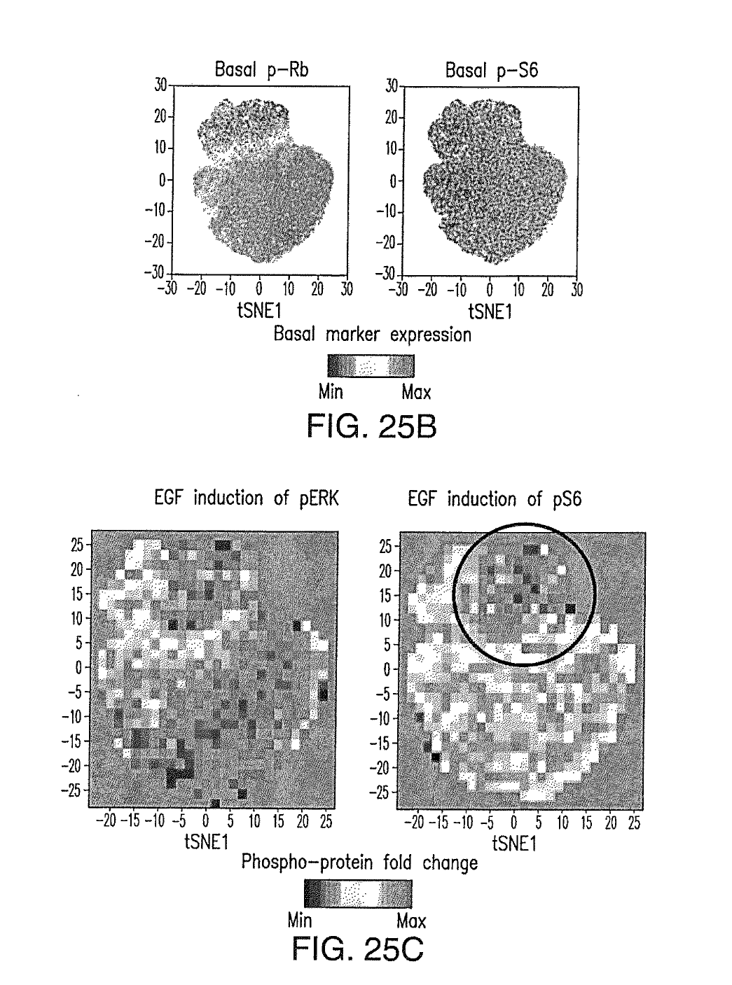

FIG. 25 shows a 32-parameter mass cytometry analysis of an adult gioblastoma xenograft with complex overlapping expression patterns of putative TIC markers and heterogeneous response to short-term EGF stimulation. (A) viSNE plot showing the single cell expression of four reported TIC markers (Sox2. CD44, a6 integrin. CD133) on 100.000 cells. The CD133 expression was scattered across eight distinct regions of the plot, suggesting eight unique patterns of co-expressed markers in the CD133+ cells. Dots represent single cells and the color corresponds to the marker expression. Axes were determined by the tSNE non-linear dimensionality reduction algorithm. which was blinded to the phospho-protein markers. (B) Basal levels of p-Rb and p-S6 were correlated with Sox2 and CD44 marker expression (compare bottom and top rows), suggesting coordinately regulated genetic programs. (C) An aliquot of the sample was treated 15 minutes ex vivo with 100 ng/mL EGF. Each small square on the heat plot represents the fold-change of the indicated marker (arcsinh transformed) in the cells in that region of the plot. While nearly all cells in the tumor responded to EGF stimulation through phosphorlation of pERK, only a subet of Sox2-CD44+CD49f+ cells responded to EGF stimulation through pS6 (in the circled region). This suggest suppression of the PI3K>Akt>mTOR>S6 signaling axis in the Sox2+ cells, perhaps through increased PTEN expression—this tumor line is known to be PTEN-wt.

### Tracking Progression Between Diagnosis and Relapse Cancer Samples

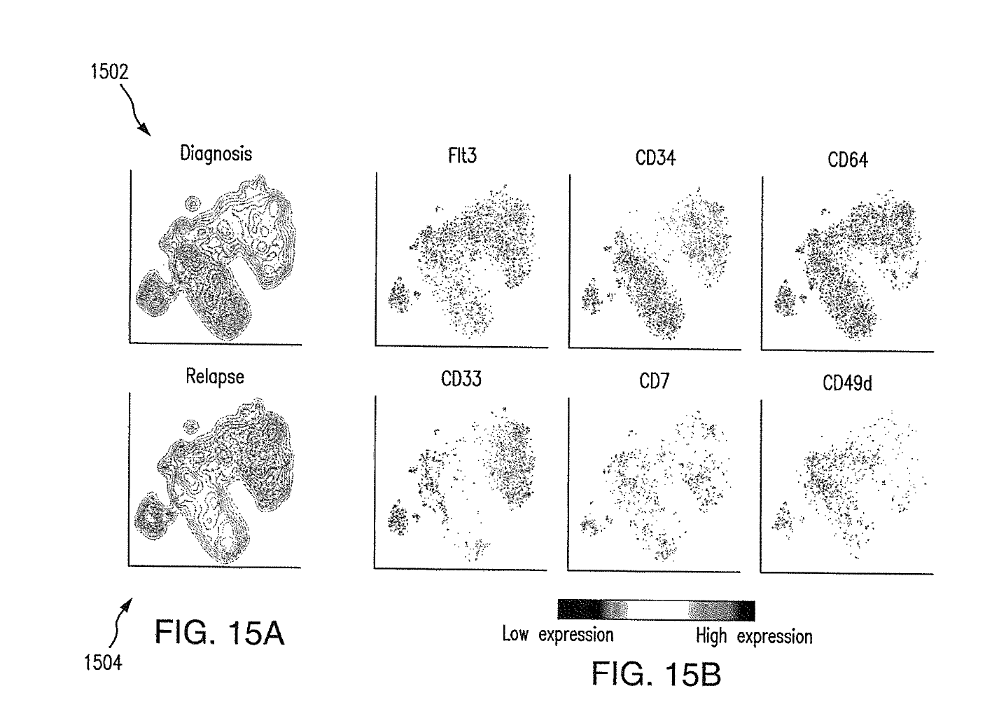

viSNE was applied to a matched diagnosis and relapse pair from a patient with AML: one sample taken before chemotherapy and another after relapse of the disease. Phenotypic states explored by the cancer were mapped and a clear separation between the diagnosis and relapse samples is seen. FIG. 15A shows contour plots of a map combining diagnosis and relapse samples. The contour lines represent cell density in each region on the map. Points represent cells from the diagnosis sample (shown in purple in map 1502) and from the relapse sample (shown in red in map 1504). Phenotypes unique to the diagnosis sample (eliminated by chemotherapy), new phenotypes developed during relapse (cancer progression), and a shared region representing phenotypes occupied by both samples (potentially indicating the source of relapse) can be identified.

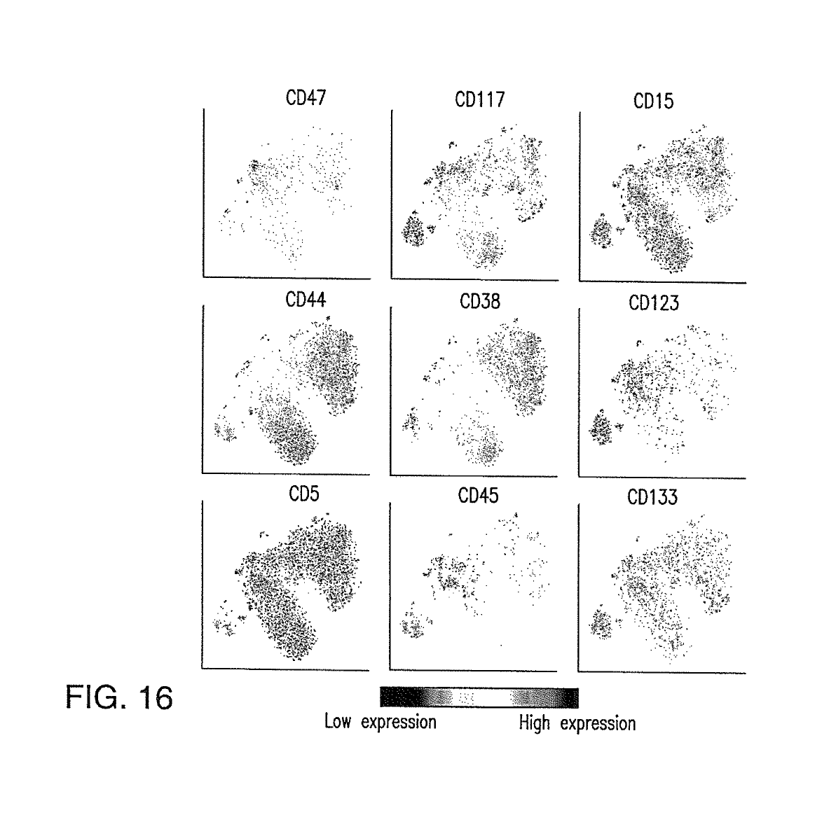

The populations of healthy cells from both the diagnosis and relapse samples that overlap in the map provide an internal control for the comparability of marker expression levels between samples. While many leukemic markers maintain equal expression levels before and after relapse, important changes emerge. With reference to FIG. 15B, a map with cells from both samples is shown. The cells are colored by marker expression levels, enabling the comparison of expression patterns before and after relapse. Genetic analysis of the diagnosis sample revealed an internal-tandem duplication of FLT3, a common mutation in AML. Flt3 expression is pervasive in the diagnosis sample, but diminished in the relapse sample, consistent with reports that Flt3 mutations can be lost between diagnosis and relapse. The cancer that reemerges at relapse has an altogether different phenotype resembling a deranged myeloid developmental program, with cells expressing both high CD34 and CD33 throughout a large fraction of the sample. The relapse sample is highly heterogeneous, distinct regions in the viSNE map express different markers from the myeloid lineage (CD64 and CD15) and lymphoid lineage (CD7). Additional maps of diagnosis and relapse AML samples, with cells colored by the expression level indicated in the panel title, are shown in FIGS. 16.

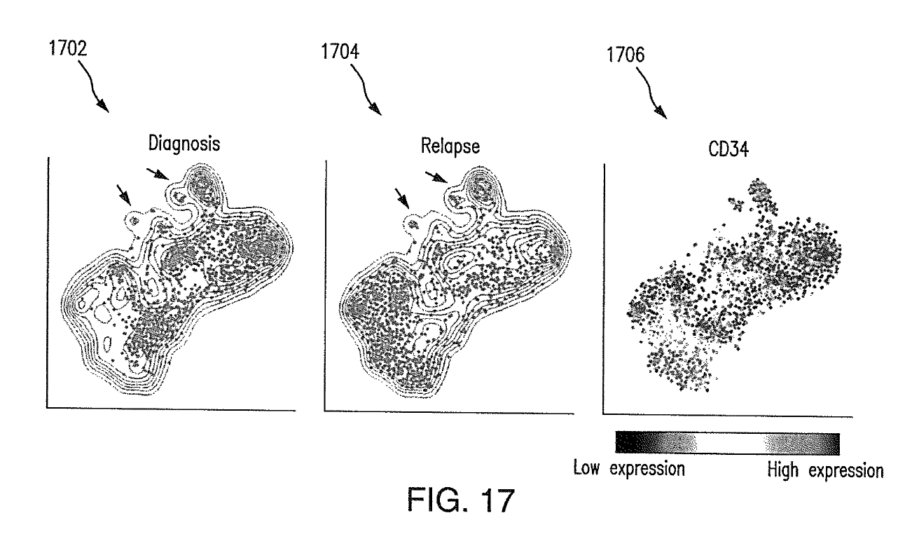

FIG. 17 shows a map of diagnosis and relapse samples of an additional patient. Contour plot 1702 shows the diagnosis cells in purple. Contour plot 1704 shows the relapse cells in red. These plots show two small healthy regions (black arrows) and a large cancer region that includes both separate and overlapping regions between diagnosis and relapse. Map 1706 shows the same data with cells colored by CD34 expression levels. A gradient of CD34 begins in the diagnosis sample and reaches its peak in the relapse region.

With further reference to FIG. 17B, exploration of the map can provide insights into the connection between diagnosis and relapse. While not intending to be bound by any particular theory of operation, the small region of the map that is populated by cells from both the diagnosis and relapse samples suggest that these might be the population from which the relapse emerged. This population is negative on CD45 and high for both CD49d and CD47, a combination reported to be expressed on stem-like chemoresistant persister cells in blood cancer, suggesting that this population is also chemoresistant. The distance and placement relative to the diagnosis sample suggests directionality of progression in which the cancer first gains stem cell markers (raising CD34) and then gains differentiation markers such as CD64 and CD7. While the map provides no conclusive evidence for any of these claims, these illustrate how exploration of the map can help raise hypotheses regarding cancer heterogeneity and its emergence.

### Minimal Residual Disease

Minimal residual disease (MRD) refers to the cancerous cells left behind after chemotherapy is completed. The presence of these cells is associated with the risk of relapse. The MRD experiment involved two samples: the MRD sample, which is composed of an in vitro mix of 99% healthy bone marrow cells and 1% bar-coded ALL cells, and the control sample, which is a 100% healthy bone marrow cells (taken from a donor other than the MRD sample's donor). To capture a higher proportion of ALL cells for the map, the following subsampling procedure was used. The cells from the MRD sample and from the control sample were combined computationally and clustered using the Louvain algorithm (described in V. D. Blondel, J. L. Guillaume, R. Lambiotte, E. Lefebvre, 2008). Next, the clusters were weighted by the proportion of MRD sample cells in them; the higher the proportion of MRD sample cells, the higher the weight. Finally, 10,000 cells were chosen one at a time in a two part process: one of the clusters was chosen randomly (biased by cluster weight) and a cell was uniformly chosen from that cluster. The subsampling procedure is blind to the ALL barcode; it can only access the mass cytometry measurement and the source of the sample (MRD or control).

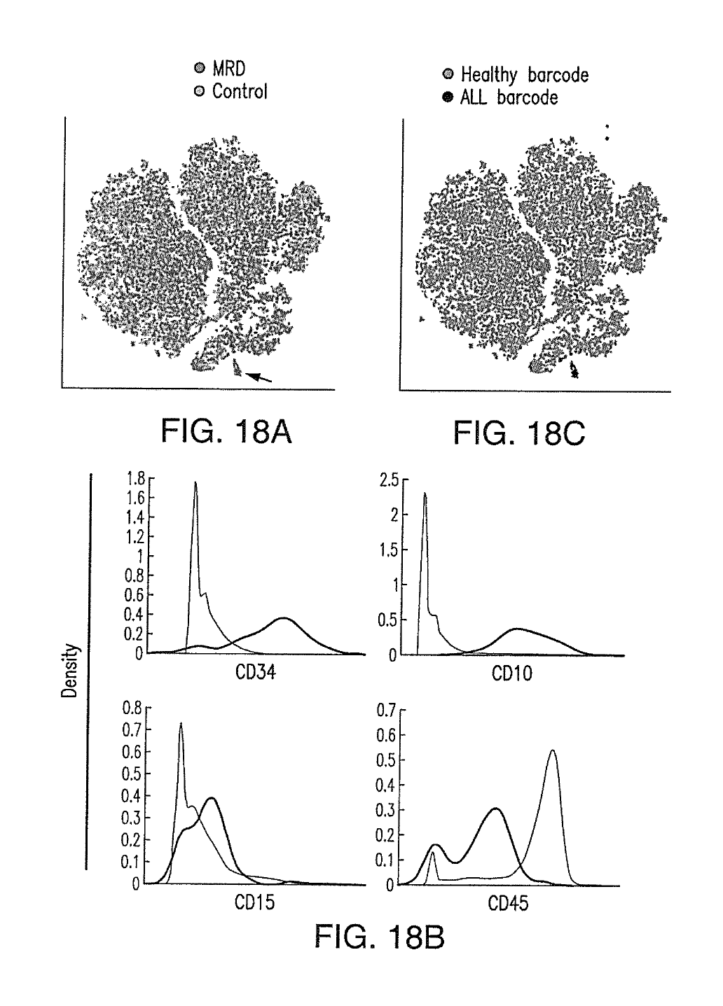

A map was generated using eight markers (CD3, CD7, CD10, CD15, CD20, CD34, CD38, CD45) to emulate a MRD scenario using fluorescence-based flow cytometry. The resulting map is illustrated in FIG. 18. In FIG. 18A, the MRD sample is shown in purple and the healthy control sample is shown in cyan. A good candidate region is distinct region of the map that contains cells from the MRD sample but no cells from the healthy control. Cells from both samples were well-mixed across most of the map except for one suspect region marked with a black arrow. Marker expression levels in the suspect region were then compared to the rest of the sample using FIG. 18B. The cells in the suspect region are labeled in red, and cells in the non-suspect region are labeled in cyan. The suspect cells strongly expressed CD10 and CD34, expressed a below-average level of CD45, and also expressed CD15 (a myeloid marker), a phenotypic combination (CD34+CD10+CD15+CD45−) often seen in B precursor ALL. Taken together, the combination of these surface markers and the absence of similar cells in the healthy control suggest that these were leukemic cells. FIG. 18C shows the map of FIG. 18A color coded by healthy barcode (cyan) and ALL barcode (red), confirming that the suspect region is entirely composed of ALL cells. While only a synthetic example, this demonstrates the ability to identify a miniscule cancer subpopulation in the data.

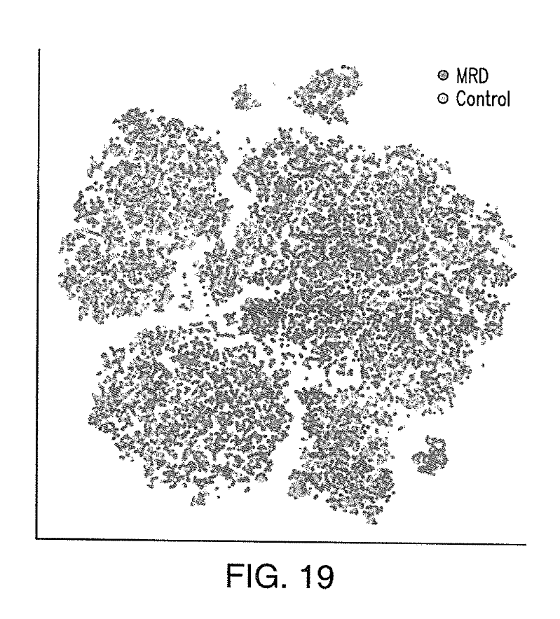

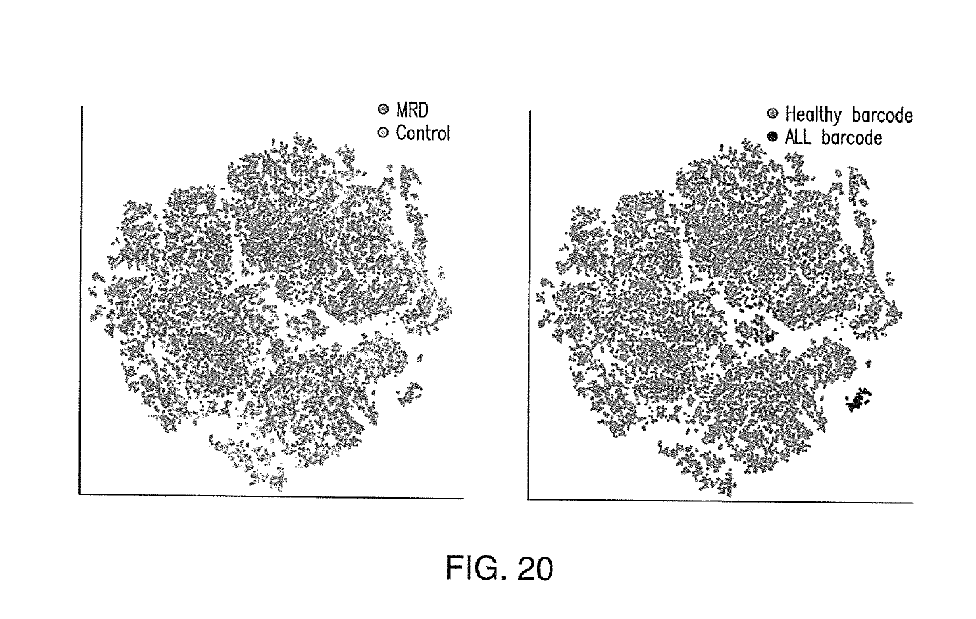

In contrast, a suspect region is not detected when only healthy cells are utilized. FIG. 19 shows a map of a healthy versus healthy run of the MRD detection procedure (i.e., using the same procedure as described above without doping the sample with ALL cells). No region with distinctly “MRD” cells exists. MRD detection also works with different channels. FIG. 20 shows the map resulting from the MRD detection procedure described above, but using a different set of eight markers. The eight channels used in the run are CD38, CD34, CD10, CD45, CD33, HLA-DR, surface IGM, and a “dump” channel that combined CD235, CD62, and CD66b. The results are similar to the results in FIG. 18.

While the present application is described herein in terms of certain preferred embodiments, those skilled in the art will recognize that various modifications and improvements can be made to the application without departing from the scope thereof. Thus, it is intended that the present application include modifications and variations that are within the scope of the appended claims and their equivalents. Moreover, it should be apparent that individual features of one embodiment can be combined with one or more features of another embodiment or features from a plurality of embodiments.

In addition to the specific embodiments claimed below, the application is also directed to other embodiments having any other combination of the dependent features claims below and those disclosed above. As such, the particular features presented in the dependent claims and disclosed above can be combined with each other in other manners within the scope of the application such that the application should be recognized as also specifically directed to other embodiments having any other combinations. Thus, the foregoing description of specific embodiments of the application has been presented for purposes of illustration and description. It is not intended to be exhaustive or to limit the application to those embodiments disclosed.

1. A computer-based method for analyzing high-dimensional single cell data based on a plurality of parameters associated with at least a first cell, comprising:
defining a point associated with the cell in a n-dimensional space, the point having n coordinates, wherein n>4;
combining said point associated with the cell with one or more other points associated with one or more other cells to form a data set;
representing each of said plurality of points in the data set in said n-dimensional space;
projecting each of said points in said n-dimensional space onto a lower-dimensional map; and
analyzing features of interest in heterogeneous tissues using said lower-dimensional map.

2. The method of claim 1, wherein the projecting further includes subsampling the data set to reduce crowding in large data sets.

3. The method of claim 1, wherein projecting further comprises using a nonlinear dimensionality reduction algorithm.

4. The method of claim 1, further comprising:
repeating a-e for at least a second cell; and
comparing the lower-dimensional map for the first cell with the lower-dimensional map of the second cell.

5. The method of claim 1, wherein the number of parameters associated with each cell corresponds to n.

6. The method of claim 1, wherein the number of parameters associated with each cell is greater than n.

7. The method of claim 6, wherein the cellular parameters utilized are chosen from one or more measured parameters according to one or more desired features of interest.

8. A computer-based system for analyzing high-dimensional single cell data based on a plurality of parameters associated with at least a first cell, comprising:
one or more memories;
one or more processors coupled to said one or more memories, where said one or more processors are configured to:
define a point associated with the cell in a n-dimensional space, the point having n coordinates, wherein n>4;
combine said point associated with the cell with one or more other points associated with one or more other cells to form in a data set;
represent each of said plurality of points in the data set in said n-dimensional space; and
project each of said points in said n-dimensional space onto a lower-dimensional map.

9. The system of claim 8, wherein said one or more processors are further configured to subsample the data set to reduce crowding in large data sets.

10. The system of claim 8, wherein said one or more processors are further configured to use a nonlinear dimensionality reduction algorithm to project said points in said n-dimensional space onto a lower-dimensional map.

11. The system of claim 8, wherein said one or more processors are further configured to:
repeat for at least a second cell; and
compare the lower-dimensional map for the first cell with the lower-dimensional map of the second cell.

12. The system of claim 8, wherein said one or more processors are further configured to obtain said plurality of parameters associated with a single cell from a measurement device that captures the relevant parameters directly.

13. The system of claim 8, wherein said one or more processors are further configured to obtain said plurality of parameters associated with a single cell from a user input device such as a keyboard.

14. The system of claim 8, wherein said one or more processors are further configured to obtain said plurality of parameters associated with a single cell from a third party via a communications network such as the Internet.

15. The system of claim 8, wherein the number of parameters associated with each cell corresponds to n.

16. The system of claim 8, wherein the number of parameters associated with each cell is greater than n.

17. The system of claim 16, wherein the cellular parameters utilized are chosen from one or more measured parameters according to one or more desired features of interest.
